(19) United States
(12) Patent Application Publication
(10) Pub. No.: US 2016/0345808 A1
(43) Pub. Date: Dec. 1, 2016

(54) ENDOSCOPE SYSTEM

(71) Applicant: OLYMPUS CORPORATION, Tokyo (JP)

(72) Inventors: Mikio INOMATA, Tokyo (JP); Kazuki HONDA, Tokyo (JP); Yasuhito KURA, Tokyo (JP)

(73) Assignee: OLYMPUS CORPORATION, Tokyo (JP)

(21) Appl. No.: 15/234,352

(22) Filed: Aug. 11, 2016

Related U.S. Application Data
(63) Continuation of application No. PCT/JP2015/056537, filed on Mar. 5, 2015.

(30) Foreign Application Priority Data
Mar. 17, 2014 (JP) 2014-054058

(51) Int. Cl. A61B 1/00; G02B 23/24; A61B 1/05

(52) U.S. Cl. CPC A61B 1/00181; A61B 1/00177; A61B 1/00009; A61B 1/00011; A61B 1/00045; A61B 1/05; G02B 23/2423; G02B 23/2484

(57) ABSTRACT

An endoscope system includes: an insertion portion; a front view observation window configured to acquire a first object image; a first image pickup device configured to generate a first image pickup signal; a side view observation window configured to acquire a second object image; a second image pickup device configured to generate a second image pickup signal; an inclination angle detection portion configured to detect a rotation around an axis and generate inclination information; and a video processor configured to generate an image signal representing a state that an image represented by the second image pickup signal is made to revolve following the rotation and arranged with respect to an image represented by the first image pickup signal, based on the inclination information, the first image pickup signal and the second image pickup signal, and output the image signal to a display portion configured to display the image.

### CROSS REFERENCE TO RELATED APPLICATION

This application is a continuation application of PCT/JP2015/056537 filed on Mar. 5, 2015 and claims benefit of Japanese Application No. 2014-054058 filed in Japan on Mar. 17, 2014, the entire contents of which are incorporated herein by this reference.

### BACKGROUND OF THE INVENTION

1. Field of the Invention

The present invention relates to an endoscope system, and in particular relates to an endoscope system capable of simultaneously observing a front view direction and a side view direction.

2. Description of the Related Art

An endoscope system including an endoscope configured to pick up an image of an object inside a subject, and an image processor configured to generate an observation image of the object picked up by the endoscope, and the like is widely used in a medical field, an industrial field, and the like.

For example, Japanese Patent Application Laid-Open Publication No. 2013-29582 discloses an in-conduit observation device including a camera with an automatic horizontal function and capable of recognizing whether or not the device is in a stable state from an inclination situation of a boundary of an image of a front direction space and an image of an upper side direction space.

In addition, Japanese Patent No. 3337682 discloses an endoscope system including an endoscope provided with a lens for front view observation configured to acquire a front view field image on a distal end face of a distal end portion of an insertion portion and provided with a plurality of lenses for side view observation configured to acquire a side view field image in a circumferential direction of the distal end portion.

The endoscope is provided with image pickup devices respectively at image-forming positions of the lens for the front view observation and the plurality of lenses for the side view observation, and picks up the front view field image and the plurality of side view field images by the image pickup devices. Then, the front view field image is arranged at a center, the plurality of side view field images are arranged at both sides of the front view field image, and the images are displayed on a monitor.

### SUMMARY OF THE INVENTION

An endoscope system of one aspect of the present invention includes an insertion portion configured to be inserted inside a subject, a first object image acquisition portion provided in the insertion portion and configured to acquire a first object image from a first region of an object inside the subject including an insertion portion front part roughly parallel to a longitudinal direction of the insertion portion, a first image pickup device configured to pick up the first object image and generate a first image pickup signal, a second object image acquisition portion provided in the insertion portion and configured to acquire a second object image from a second region of the object different from the first region, the second region including an insertion portion side part in a direction crossing the longitudinal direction of the insertion portion, a second image pickup device configured to pick up the second object image and generate a second image pickup signal, an inclination detection portion configured to detect a rotation around an axis, with the axis being the longitudinal direction of the insertion portion, and generate inclination information, and an image signal generation portion configured to generate an image signal representing a state that an image represented by the second image pickup signal is made to revolve following the rotation and arranged with respect to an image represented by the first image pickup signal, based on the inclination information, the first image pickup signal and the second image pickup signal, and output the image signal to a display portion configured to display the image.

### BRIEF DESCRIPTION OF THE DRAWINGS

FIG. 1 is a diagram illustrating a configuration of an endoscope system relating to a first embodiment;

FIG. 2 is a perspective view illustrating a configuration of a distal end portion of an insertion portion of an endoscope;

FIG. 3 is a sectional view illustrating a cross section of the distal end portion of the insertion portion of the endoscope;

FIG. 4 is a diagram illustrating a configuration of a principal part in the first embodiment;

FIG. 5 is a diagram illustrating a configuration of the principal part in the first embodiment;

FIG. 6A is a diagram illustrating one example of an observation image displayed on a monitor by image processing by an image processing portion 32a;

FIG. 6B is a diagram illustrating one example of an observation image displayed on a monitor by image processing by the image processing portion 32a;

FIG. 6C is a diagram illustrating one example of an observation image displayed on a monitor by image processing by the image processing portion 32a;

FIG. 7 is a diagram for illustrating another example of an observation image generated in the image processing portion 32a;

FIG. 8 is a diagram for illustrating another example of an observation image generated in the image processing portion 32a;

FIG. 9 is a diagram for illustrating another example of an observation image generated in the image processing portion 32a;

FIG. 10 is a diagram for illustrating another example of an observation image generated in the image processing portion 32a;

FIG. 11 is a perspective view illustrating a configuration of a distal end portion of an insertion portion of an endoscope relating to a second embodiment;

FIG. 12 is a front view illustrating a configuration of the distal end portion of the insertion portion of the endoscope relating to the second embodiment;

FIG. 13 is a sectional view of the insertion portion relating to the second embodiment;

FIG. 14 is a diagram illustrating a configuration of a principal part in the second embodiment;

FIG. 15 is a diagram illustrating a configuration of the principal part in the second embodiment;

FIG. 16A is a diagram illustrating one example of an observation image displayed on a monitor by image processing by an image processing portion 32a1;

FIG. 16B is a diagram illustrating one example of an observation image displayed on a monitor by image processing by the image processing portion 32a1;

FIG. 16C is a diagram illustrating one example of an observation image displayed on a monitor by image processing by the image processing portion 32a1;

FIG. 17 is a diagram illustrating a configuration of a principal part in a third embodiment;

FIG. 18 is a diagram illustrating a configuration of a principal part in a fourth embodiment;

FIG. 19 is a diagram illustrating a configuration of a principal part in a fifth embodiment;

FIG. 20 is a diagram for illustrating a rotating operation of a monitor 35; and

FIG. 21 is a perspective view of a distal end portion 6 of an insertion portion 4 to which a unit for side observation is attached.

### DETAILED DESCRIPTION OF THE PREFERRED EMBODIMENT(S)

Hereinafter, embodiments of the present invention will be described with reference to the drawings.

### First Embodiment

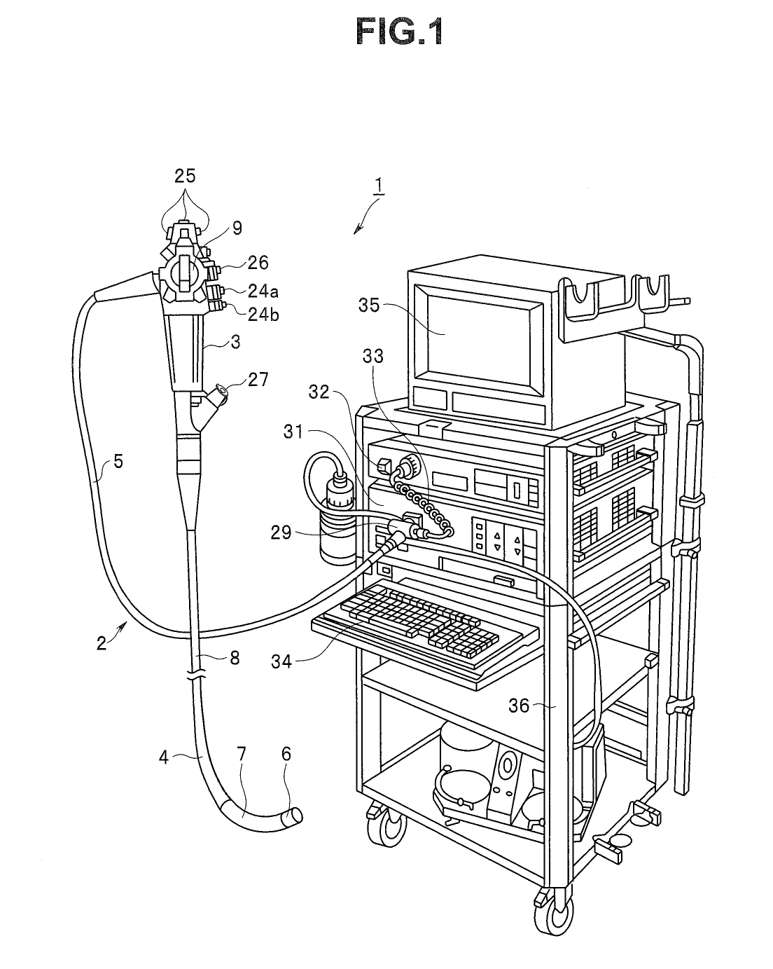

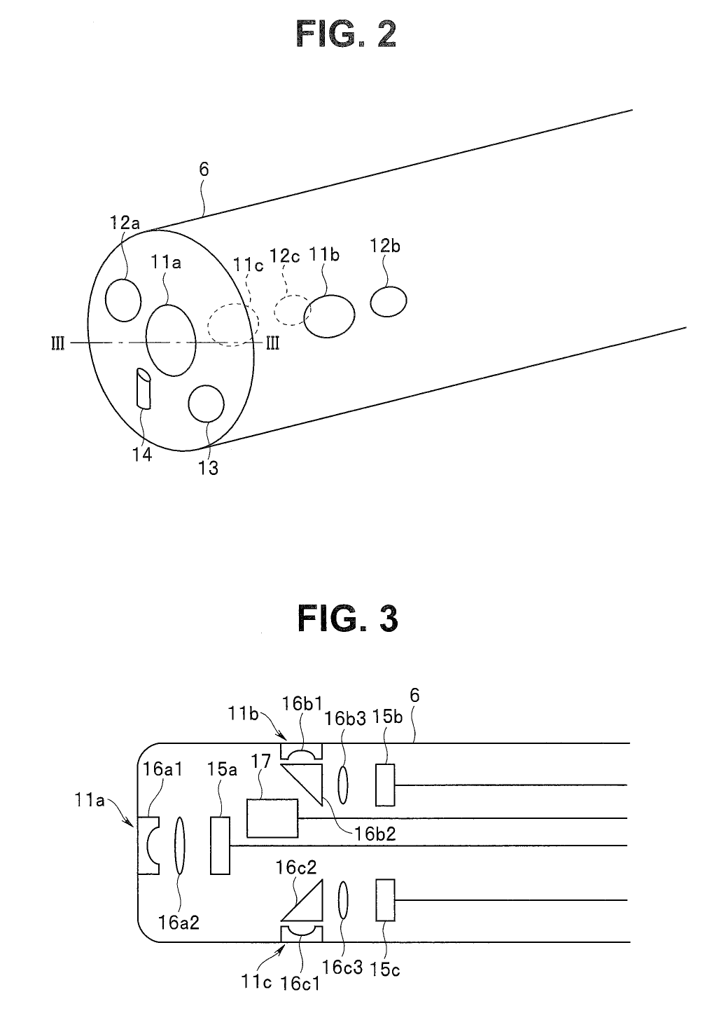

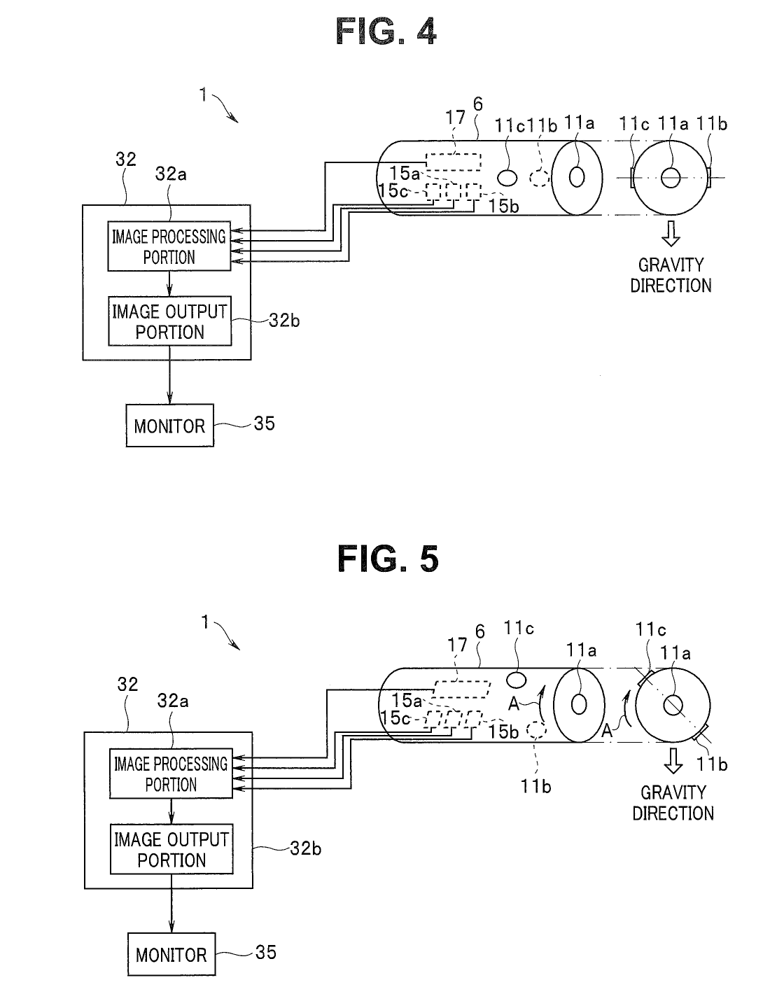

First, a configuration of an endoscope system of the first embodiment will be described using FIG. 1 to FIG. 5. FIG. 1 is a diagram illustrating the configuration of the endoscope system relating to the first embodiment, FIG. 2 is a perspective view illustrating a configuration of a distal end portion of an insertion portion of an endoscope, FIG. 3 is a sectional view illustrating a cross section of the distal end portion of the insertion portion of the endoscope, and FIG. 4 and FIG. 5 are diagrams illustrating a configuration of a principal part in the first embodiment.

As illustrated in FIG. 1, an endoscope system 1 includes an endoscope 2 configured to pick up an image of an observation object and output an image pickup signal, a light source device 31 configured to supply illuminating light for illuminating the observation object, a video processor 32 having a function as an image signal generation portion configured to generate and output a video signal (image signal) according to the image pickup signal, and a monitor 35 configured to display an observation image according to the video signal (image signal).

The endoscope 2 is configured including an operation portion 3 configured to be held by an operator to perform operations, an elongated insertion portion 4 formed on a distal end side of the operation portion 3 and configured to be inserted into a body cavity or the like, and a universal cord 5 one end of which is provided so as to extend from a side part of the operation portion 3.

The endoscope 2 of the present embodiment is a wide angle endoscope capable of observing a visual field of 180 degrees or wider by displaying a plurality of field images, and prevents overlooking of a lesion at a location where the lesion is hard to see just by observation in a front view direction, such as a back of folds, a boundary of organs, and the like, inside a body cavity, in particular inside a large intestine. When inserting the insertion portion 4 of the endoscope 2 into the large intestine, an operation of temporary fixation or the like by performing twisting, reciprocating motions and hooking of an intestinal wall with the insertion portion 4 is generated similarly to a normal colonoscope.

The insertion portion 4 is configured including a hard distal end portion 6 provided on a most distal end side, a freely bendable bending portion 7 provided on a rear end of the distal end portion 6, and a flexible tube portion 8 which has a long length and flexibility and is provided on a rear end of the bending portion 7. In addition, the bending portion 7 performs a bending operation according to an operation of a bending operation lever 9 provided on the operation portion 3.

In the meantime, as illustrated in FIG. 2, a front view observation window 11a for observing a front view direction is arranged on a distal end face of the distal end portion 6 of the endoscope 2, and a plurality (two, in the example of FIG. 2) of side view observation windows 11b and 11c for observing a side view direction are arranged on a side face of the distal end portion 6 of the endoscope 2. The side view observation windows 11b and 11c are arranged at an equal interval in a circumferential direction of the distal end portion 6, for example, at an interval of 180 degrees. Note that the number of the side view observation windows 11b and 11c arranged at the equal interval in the circumferential direction of the distal end portion 6 is not limited to two, and a configuration may be such that one, or three or more side view observation windows are arranged for example.

On the distal end face of the distal end portion 6 of the endoscope 2, at a position adjacent to the front view observation window 11a, at least one front view illumination window 12a configured to emit illuminating light in a range of a front view field of the front view observation window 11a is arranged. Also, on the side face of the distal end portion 6 of the endoscope 2, at positions adjacent to the respective side view observation windows 11b and 11c, at least one side view illumination windows 12b and 12c configured to emit illuminating light in a range of a side view field of the side view observation windows 11b and 11c is arranged.

In addition, on the distal end face of the distal end portion 6 of the endoscope 2, a distal end opening portion 13 communicated with a treatment instrument channel formed by a tube or the like inside the insertion portion 4 and not shown in the figure, and capable of projecting a (distal end portion of) treatment instrument inserted to the treatment instrument channel, and a nozzle portion 14 for the front view observation window configured to eject a gas or liquid for cleaning the front view observation window 11a are provided. Further, on the side face of the distal end portion 6 of the endoscope 2, the nozzle portion for the side view observation window configured to eject a gas or liquid for cleaning the side view observation windows 11b and 11c and not shown in the figure is provided adjacently to each of the side view observation windows 11b and 11 c.

The operation portion 3 is provided with, as illustrated in FIG. 1, an air supply and liquid supply operation button 24a capable of instructing an operation of causing the gas or liquid for cleaning the front view observation window 11a to be ejected from the nozzle portion 14 for the front view observation window, and an air supply and liquid supply operation button 24b capable of instructing an operation of causing the gas or liquid for cleaning the side view observation windows 11b and 11c to be ejected from the nozzle portion 14 for the side view observation window not shown in the figure, and air supply and liquid supply can be switched by depression of the air supply and liquid supply operation buttons 24a and 24b. In addition, in the present embodiment, the plurality of air supply and liquid supply operation buttons are provided so as to correspond to the respective nozzle portions; however, the gas or liquid may be ejected from both of the nozzle portion 14 for the front view observation window and the nozzle portion for the side view observation window not shown in the figure by the operation of one air supply and liquid supply operation button for example.

A plurality of scope switches 25 are provided on a peak of the operation portion 3, and have a configuration that functions for the respective switches can be allocated so as to cause signals corresponding to ON or OFF or the like of various descriptions usable in the endoscope 2 to be outputted. Specifically, to the scope switches 25, for example, functions of causing signals corresponding to start and stop of forward water supply, execution and cancellation of freeze, and notification of a using state of a treatment instrument, and the like to be outputted can be allocated as the functions for the respective switches.

Note that, in the present embodiment, a function of at least either one of the air supply and liquid supply operation buttons 24a and 24b may be allocated to one of the scope switches 25.

Also, at the operation portion 3, a suction operation button 26 capable of giving an instruction for sucking and recovering mucus or the like inside a body cavity from the distal end opening portion 13 to a suction unit or the like not shown in the figure is disposed.

Then, the mucus or the like inside the body cavity sucked according to the operation of the suction unit or the like not shown in the figure is made to pass through the distal end opening portion 13, the treatment instrument channel not shown in the figure inside the insertion portion 4, and a treatment instrument insertion port 27 provided near a front end of the operation portion 3, and is then recovered to a suction bottle or the like of the suction unit not shown in the figure.

The treatment instrument insertion port 27 is communicated with the treatment instrument channel not shown in the figure inside the insertion portion 4, and is formed as an opening to which a treatment instrument not shown in the figure can be inserted. That is, an operator can perform a treatment using the treatment instrument by inserting the treatment instrument from the treatment instrument insertion port 27 and projecting a distal end side of the treatment instrument from the distal end opening portion 13.

In the meantime, as illustrated in FIG. 1, on the other end of the universal cord 5, a connector 29 connectable to the light source device 31 is provided.

On a distal end portion of the connector 29, a pipe sleeve (not shown in the figure) to be a connection end of a fluid pipeline and a light guide pipe sleeve (not shown in the figure) to be a supply end of the illuminating light are provided. In addition, on a side face of the connector 29, an electric contact portion (not shown in the figure) capable of connecting one end of a connection cable 33 is provided. Further, on the other end of the connection cable 33, a connector for electrically connecting the endoscope 2 and the video processor 32 is provided.

The universal cord 5 incorporates a plurality of signal lines for transmitting various electric signals and a light guide for transmitting the illuminating light supplied from the light source device 31 in a bound state.

The light guide incorporated from the insertion portion 4 to the universal cord 5 has the configuration that an end on a light emission side is branched into at least three directions near the insertion portion 4, and respective light emission end face is arranged at the front view illumination window 12a and the side view illumination windows 12b and 12c as light emission portions. In addition, the light guide has the configuration that an end on a light incidence side is arranged at the light guide pipe sleeve of the connector 29.

Note that the light emission portions arranged at the front view illumination window 12a and the side view illumination windows 12b and 12c may be a light emitting element like a light emitting diode (LED) instead of the light guide.

The video processor 32 outputs drive signals for driving a plurality of image pickup devices provided on the distal end portion 6 of the endoscope 2. Then, the video processor 32 functions as an image signal generation portion configured to generate video signals (image signals) and output the video signals to the monitor 35 by executing signal processing to image pickup signals outputted from the plurality of image pickup devices. While details are described later, the video processor 32 arranges a front view field image acquired at the front view observation window 11a at a center, arranges two side view field images acquired at the side view observation windows 11b and 11c on left and right of the front view field image, executes predetermined image processing to the front view field image and the two side view field images, and outputs the images to the monitor 35. That is, the video processor 32 performs a treatment so that the front view field image acquired at the front view observation window 11a and the side view field images acquired at the side view observation windows 11b and 11c are arranged side by side in an adjacent state, and generates the video signals.

Peripheral devices such as the light source device 31, the video processor 32 and the monitor 35 are arranged at a frame 36 together with a keyboard 34 configured to perform input of patient information or the like.

As illustrated in FIG. 3, the front view observation window 11a configuring a first object image acquisition portion acquires a first object image from a front view direction (first direction) including a front part roughly parallel to a longitudinal direction of the insertion portion 4, that is, a first region of an object. The front view observation window 11a includes an objective optical system 16a1, an image pickup device 15a is arranged at an image forming position of the front view observation window 11a and the objective optical systems 16a1 and 16a2, and the object image acquired at the front view observation window 11a is photoelectrically converted. Note that the distal end portion 6 illustrated in FIG. 3 is a sectional view of a line in FIG. 2.

In addition, the side view observation window (at least one or more side view observation windows of the side view observation windows 11b and 11c) configuring a second object image acquisition portion acquires a second object image from a side view direction (second direction) including a direction crossing the longitudinal direction of the insertion portion at least partially different from the front view direction (first direction), that is, a second region of the object.

The side view observation window 11b includes an objective optical system 16b1, an image pickup device 15b is arranged at an image forming position of the side view observation window 11b and the objective optical systems 16b1, 16b2 and 16b3, and the object image acquired at the side view observation window 11b is photoelectrically converted.

Similarly, the side view observation window 11c includes an objective optical system 16c1, an image pickup device 15c is arranged at an image forming position of the side view observation window 11c and the objective optical systems 16c1, 16c2 and 16c3, and the object image acquired at the side view observation window 11c is photoelectrically converted.

Note that the image pickup device 15b may be arranged so as to directly face the objective optical system 16b1 without interposing the objective optical systems 16b2 and 12b3. Similarly, the image pickup device 15c may be arranged so as to directly face the objective optical system 16c1 without interposing the objective optical systems 16c2 and 16c3.

Boundary regions of the first object image and the second object image may overlap or may not overlap, and in a state that the boundary regions overlap, the first object image acquisition portion and the second object image acquisition portion may acquire partially overlapping object images, and processing of partially removing an overlapping region may be performed in the video processor 32.

In addition, on a rear end side of the image pickup device 15a of the distal end portion 6, an inclination angle detection portion 17 is provided. The inclination angle detection portion 17 as an inclination detection portion detects an inclination angle of a rotating direction around an axis to a gravity direction of the insertion portion 4, with the axis being the longitudinal direction of the insertion portion 4, and generates inclination angle information (inclination information). As illustrated in FIG. 5, the inclination angle detection portion 17 detects the inclination angle of the rotating direction around the axis to the gravity direction of the insertion portion 4, with the axis being the longitudinal direction of the insertion portion 4, when a user twists the insertion portion 4 (or, the insertion portion 4 is naturally twisted while being inserted into the body cavity or the like) for instance. Note that the inclination angle detection portion 17 is configured by an acceleration sensor or an angular velocity sensor (gyro sensor) for example, but is not limited to the sensors as long as the inclination angle of the rotating direction around the axis to the gravity direction of the insertion portion 4 is detected.

As illustrated in FIG. 4, the image pickup devices 15a-15c are electrically connected to an image processing portion 32a operated also as the image signal generation portion respectively, and output the front view field image picked up in the image pickup device 15a and the side view field images picked up in the respective image pickup devices 15b and 15c to the image processing portion 32a. Also, the inclination angle detection portion 17 is electrically connected to the image processing portion 32a, and outputs the detected inclination angle information (inclination information) to the image processing portion 32a.

The image processing portion 32a generates the front view field image from the object image acquired at the front view observation window 11a and arranges the image at the center, generates the side view field images respectively from the two object images acquired at the side view observation windows 11b and 11c and arranges the images on the left and right of the front view field image, also executes the predetermined image processing to the front view field image and the two side view field images, and outputs the images to an image output portion 32b. Specifically, the image processing portion 32a executes image processing of rotating the front view field image and the side view field images arranged on the left and right following the inclination angle, based on the information of the inclination angle of the insertion portion 4 of the endoscope 2 detected by the inclination angle detection portion 17.

The image output portion 32b generates signals to be displayed on the monitor 35 from the image signals generated by the image processing portion 32a, and outputs the signals to the monitor 35.

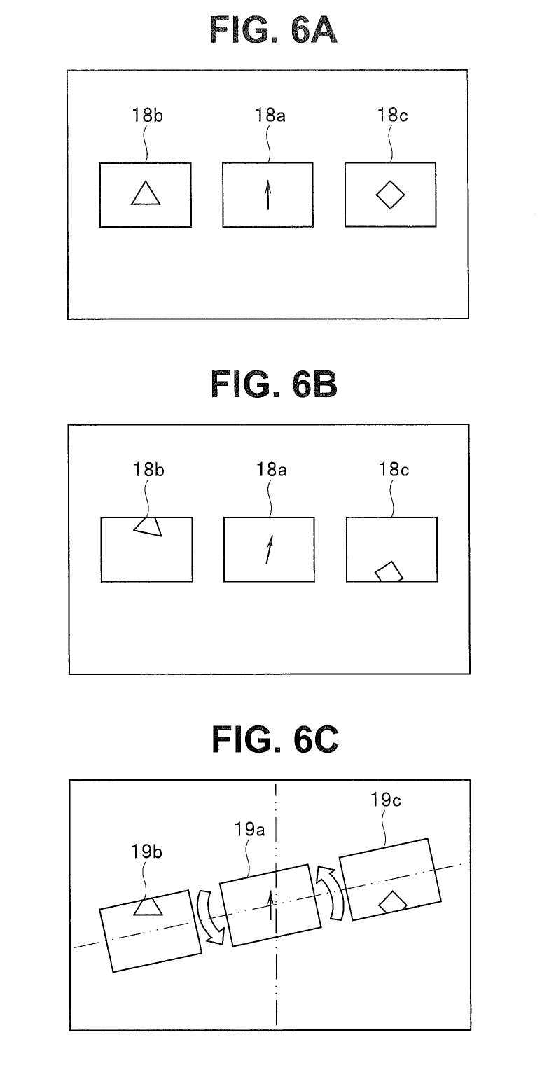

Next, the image processing by the image processing portion 32a will be described using FIG. 6A to FIG. 6C. FIG. 6A to FIG. 6C are diagrams illustrating one example of observation images displayed on the monitor by the image processing by the image processing portion 32a.

The image processing portion 32a acquires a front view field image 18a based on the first object image acquired at the front view observation window 11a, and side view field images 18b and 18c based on the second object image acquired at the side view observation windows 11b and 11c. Then, the image processing portion 32a, as illustrated in FIG. 6A, arranges the front view field image 18a at the center and arranges the side view field images 18b and 18c on the left and right of the front view field image 18a. Specifically, the image processing portion 32a arranges the side view field image 18b on a left side of the front view field image 18a, and arranges the side view field image 18c on a right side of the front view field image 18a.

Here, when the insertion portion 4 of the endoscope 2 is twisted (when a twisting operation is performed) as illustrated in FIG. 5, as illustrated in FIG. 6B, view points of the image pickup devices 15a-15c provided on the distal end portion 6 of the insertion portion 4 are also rotated around the axis in the longitudinal direction of the insertion portion 4 and an observation image is rotated so that it becomes difficult for a user to perform observation.

Therefore, the image processing portion 32a generates a front view field image 19a and side view field images 19b and 19c for which the front view field image 18a and side view field images 18b and 18c are rotated following the inclination angle of the insertion portion 4 as illustrated in FIG. 6C, based on the inclination angle information (inclination information) from the inclination angle detection portion 17.

In such a manner, the image processing portion 32a generates the image signals representing the state that an arrangement angle of a direction where the visual field images are arranged is changed and the first object image and the second object images are inclined by attaining an arrangement relationship of adjacently arranging the second object images acquired at the side view observation windows 11b and 11c at parts including both sides of the first object image acquired at the front view observation window 11a and relatively rotating the second object images to the arrangement of the first object image based on the inclination angle information (inclination information).

Note that, though details will be described later, the image processing portion 32a may generate the image signals representing the state that only the second object images are inclined, based on the inclination angle information (inclination information).

In addition, in the case of displaying a plurality of images on the monitor 35, the configuration is such that the side view field images 19b and 19c are arranged on the left and right of the front view field image 19a; however, without being limited to the configuration, it is sufficient when the front view field image and the side view field images are adjacent, and the configuration may be such that the side view field images are arranged on either left or right side of the front view field image 19a.

Also, in the present embodiment, the plurality of images are displayed on the monitor 35; however, it is not limited to the manner. For example, the configuration may be such that three monitors are adjacently arranged, the front view field image 19a is displayed on the monitor at the center, and the side view field images 19b and 19c are displayed respectively on the monitors on the left and right.

In such a manner, the endoscope system 1 acquires the front view field image 18a by the front view observation window 11a, acquires the side view field images 18b and 18c by the side view observation windows 11b and 11c, arranges the front view field image 18a at the center, and arranges the side view field images 18b and 18c on the left and right of the front view field image 18a. Then, the endoscope system 1 generates the front view field image 19a and the side view field images 19b and 19c for which the front view field image 18a and the side view field images 18b and 18c are rotated based on the inclination angle information (inclination information) from the inclination angle detection portion 17.

As a result, since the front view field image 19a and the side view field images 19b and 19c which are rotated according to a rotating operation of the insertion portion 4 are displayed on the monitor 35, a user such as an operator can recognize the inclination and rotating angle or the like of the insertion portion 4.

Thus, according to the endoscope system of the present embodiment, a range of performing the observation when the rotating operation of the endoscope or the like is performed can be easily recognized.

Thus, even in the case that it is difficult to recognize a posture (inclination, rotating angle) of the insertion portion at present just by contents of the images displayed as the front view field image and the side view field images, by attaining the configuration of changing the arrangement angle of the direction where the field images are arranged as in the endoscope system of the present embodiment, a user such as an operator can more quickly and intuitively recognize the posture of the insertion portion at present also from the arrangement of the field images.

For example, during a screening inspection of inserting the insertion portion of an endoscope deep inside a subject and thoroughly confirming a side view field while slowly removing the insertion portion, in the case that the insertion portion is rotated even though a target part is found within the side view field immediately before and the target part becomes invisible for example, it becomes easy for a user to return the insertion portion in the rotating direction and perforin an inspection again for instance based on the arrangement of the side view field images.

By the configuration, it becomes possible to more appropriately recognize that a part different in the rotating direction at present from a previously observed side view field is being observed even when an insertion amount of the insertion portion is the same, and it is possible to prevent a possibility of losing a location observed at present and overlooking the target part in the rotating direction.

### (Modification)

Note that the image processing portion 32a may not only perform the image processing of rotating the front view field image 18a and the side view field images 18b and 18c based on the inclination angle information (inclination information) from the inclination angle detection portion 17 but also perform image processing as follows.

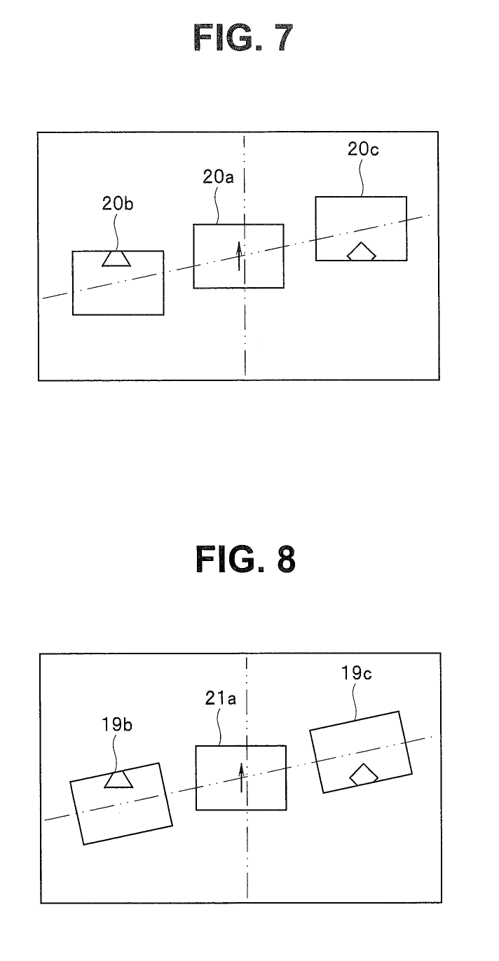

FIG. 7 to FIG. 10 are diagrams for describing other examples of observation images generated in the image processing portion 32a.

The image processing portion 32a generates the front view field image 19a and side view field images 19b and 19c for which the front view field image 18a and side view field images 18b and 18c are rotated according to the inclination angle information (inclination information) from the inclination angle detection portion 17.

Thereafter, the image processing portion 32a, as illustrated in FIG. 7, generates a front view field image 20a and side view field images 20b and 20c for which rectangular masks are executed to the front view field image 19a and side view field images 19b and 19c and causes the images to be displayed on the monitor 35. That is, a display mode in which the side view field images 19b and 19c revolve with respect to the front view field image 19a is attained. Thus, an effect similar to that of the embodiment described above can be obtained.

In addition, the image processing portion 32a generates the front view field image 19a and side view field images 19b and 19c for which the front view field image 18a and side view field images 18b and 18c are rotated according to the inclination angle information (inclination information) from the inclination angle detection portion 17.

Thereafter, the image processing portion 32a, as illustrated in FIG. 8, generates a front view field image 21a and side view field images 20b and 20c for which rectangular masks are executed to the front view field image 19a and side view field images 19b and 19c, rotates the rectangular masks to be frames of displaying the side view field images 19b and 19c and causes the images to be displayed on the monitor 35. That is, a display mode in which the side view field images 19b and 19c (the frames of displaying the side view field images 19b and 19c) themselves revolve with respect to the front view field image 19a while rotating is attained. Thus, an effect similar to that of the embodiment described above can be obtained.

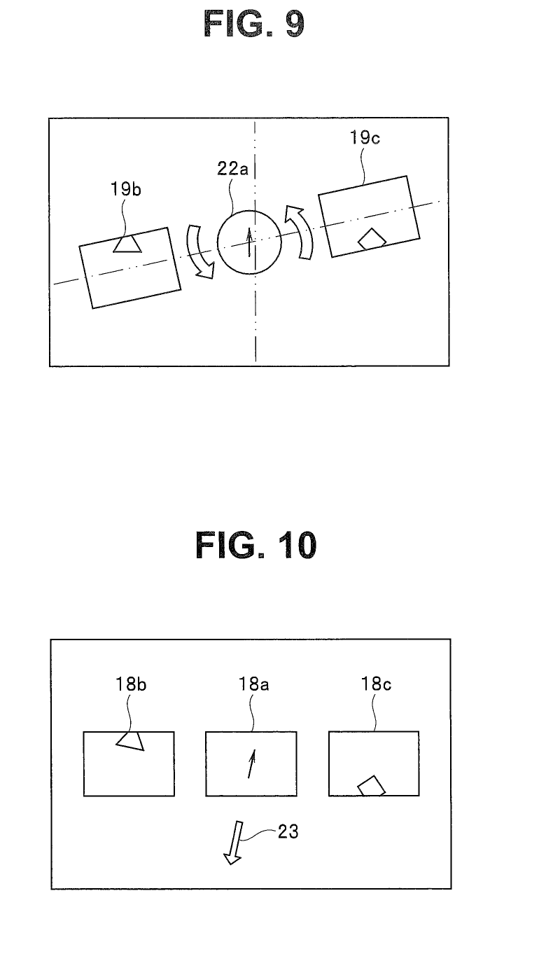

In addition, the image processing portion 32a generates the front view field image 19a and side view field images 19b and 19c for which the front view field image 18a and side view field images 18b and 18c are rotated according to the inclination angle information (inclination information) from the inclination angle detection portion 17. Thereafter, the image processing portion 32a, as illustrated in FIG. 9, generates a front view field image 22a for which a round mask is executed only to the front view field image 19a and causes the images to be displayed on the monitor 35. By generating the front view field image 22a for which the round mask is executed only to the front view field image 19a in such a manner, an effect similar to that of the embodiment described above can be obtained, and it is made easy for a user to recognize that the side view field images 19b and 19c are rotating centering on the front view field image 22a.

Note that the respective display modes illustrated in FIG. 6C to FIG. 9 may be appropriately switched.

In addition, the image processing portion 32a arranges the front view field image 18a and the side view field images 18b and 18c side by side and then, generates an index 23 indicating an inclination amount of the insertion portion 4. Then, the image processing portion 32a, as illustrated in FIG. 10, rotates the index 23 following the inclination angle of the insertion portion 4 based on the inclination angle information (inclination information) from the inclination angle detection portion 17, and causes the index to be displayed on the monitor 35. Thus, an effect similar to that of the embodiment described above can be obtained.

By using the index 23 in combination with the respective embodiments of the first embodiment, a user can more surely recognize the posture of the insertion portion 4.

### Second Embodiment

Next, the second embodiment will be described.

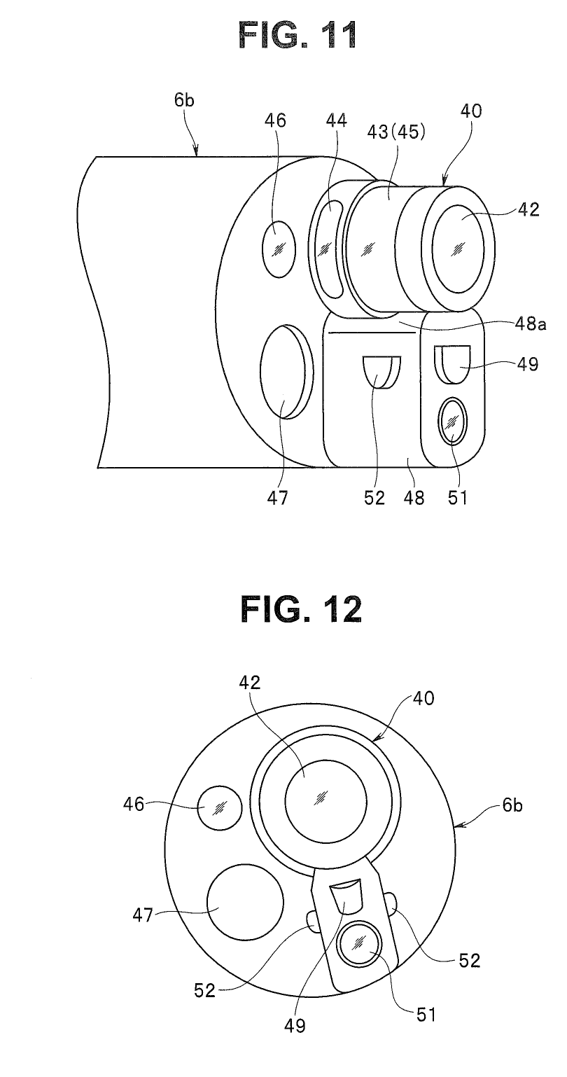

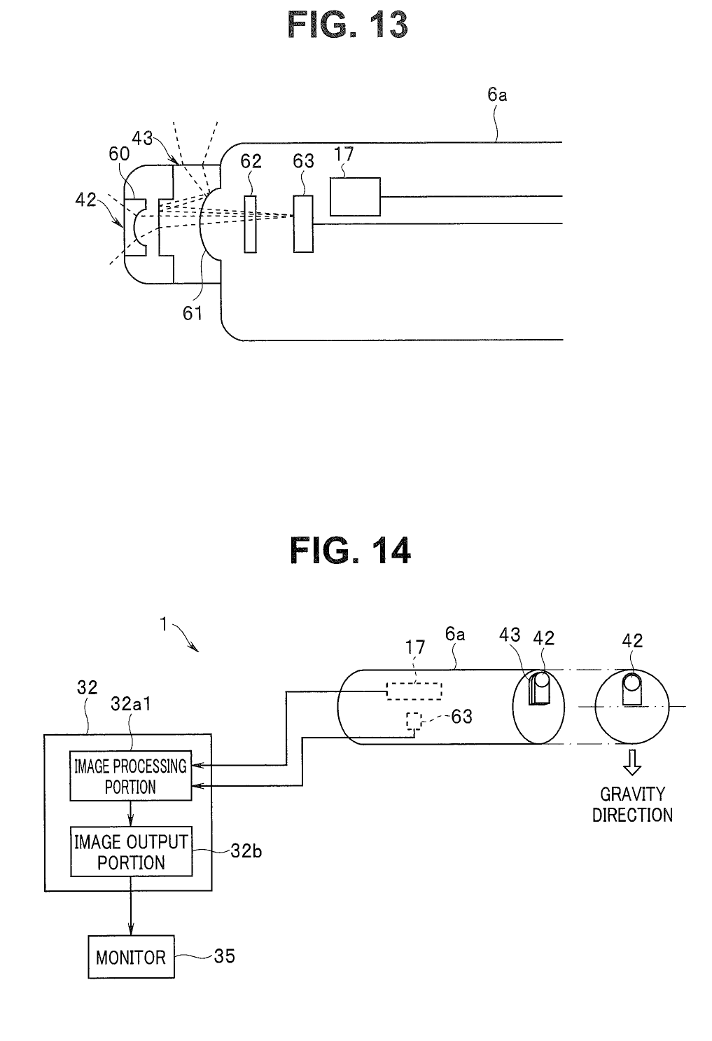

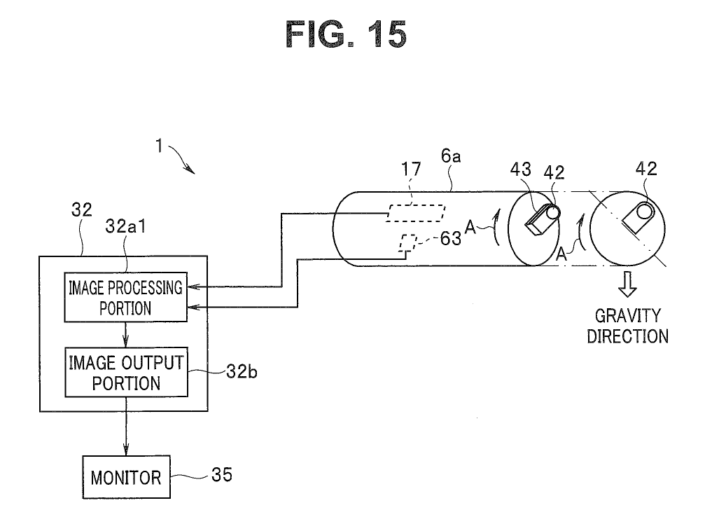

FIG. 11 is a perspective view illustrating a configuration of a distal end portion of an insertion portion of an endoscope relating to the second embodiment, FIG. 12 is a front view illustrating the configuration of the distal end portion of the insertion portion of the endoscope relating to the second embodiment, FIG. 13 is a sectional view of the insertion portion relating to the second embodiment, and FIG. 14 and FIG. 15 are diagrams illustrating a configuration of a principal part in the second embodiment.

As illustrated in FIG. 11, at a distal end portion 6b of the insertion portion 4, a cylindrical portion 40 in a columnar shape provided by being projected from a position biased upwards from the center of the distal end face of the distal end portion 6b is formed.

On the distal end portion of the cylindrical portion 40, an objective optical system 62 serving for both front view and side view is provided as illustrated in FIG. 13. In addition, the distal end portion of the cylindrical portion 40 is configured including a front view observation window 42 configuring the first object image acquisition portion arranged at a part corresponding to the front view direction of an objective optical system 60, and a side view observation window 43 configuring the second object image acquisition portion arranged at a part corresponding to the side view direction of an objective optical system 61.

The front view observation window 42 acquires the first object image from the front view direction (first direction) including the front part roughly parallel to the longitudinal direction of the insertion portion 4, that is, the first region of the object.

In addition, the side view observation window 43 acquires the second object image from the side view direction (second direction) including the direction crossing the longitudinal direction of the insertion portion at least partially different from the front view direction (first direction), that is the second region of the object.

Further, near a proximal end of the cylindrical portion 40, a side view illumination portion 44 configured to emit light for illuminating the side view direction is formed.

The side view observation window 43 includes a mirror lens 45 for the side view to be capable of acquiring the side view field image by capturing return light (reflected light) from the observation object made incident from the circumferential direction in the cylindrical portion 40 in the columnar shape within the side view field.

Note that, at an image forming position of the objective optical system 62, (an image pickup surface of) an image pickup device 63 illustrated in FIG. 13 is arranged so as to form the image of the observation object within the visual field of the front view observation window 42 at a center part as a circular front view field image, and to form the image of the observation object within the visual field of the side view observation window 43 at an outer peripheral part of the front view field image as the side view field image in a circular ring shape.

Such an image is achieved by using a two-time reflection optical system configured to reflect the return light two times by the mirror lens for the side view; however, the image may be formed by reflecting the return light once by a one-time reflection optical system, and the image may be processed in the video processor 32 to match directions of the side view field image and the front view field image.

Also, on a rear end side of the image pickup device 63 at the distal end portion 6b, the inclination angle detection portion 17 configured to detect the inclination angle of the rotating direction around the axis to the gravity direction of the insertion portion 4, with the axis being the longitudinal direction of the insertion portion 4, and generate the inclination angle information (inclination information), similarly to the first embodiment.

On the distal end face of the distal end portion 6b, a front view illumination window 46 arranged at a position adjacent to the cylindrical portion 40 and configured to emit the illuminating light in the range of the front view field of the front view observation window 42, and a distal end opening portion 47 communicated with the treatment instrument channel formed by a tube or the like inside the insertion portion 4 and not shown in the figure, and capable of projecting a (distal end portion of) treatment instrument inserted to the treatment instrument channel are provided.

Means of supplying the illuminating light to the side view illumination portion 44 and the front view illumination window 46 may be a method of guiding the illuminating light from a light source by a light guide or may be illumination from a light emitting element like a light emitting diode (LED) installed nearby.

In addition, the distal end portion 6b of the insertion portion 4 includes a support portion 48 provided so as to be projected from the distal end face of the distal end portion 6b, and the support portion 48 is positioned adjacently to a lower side of the cylindrical portion 40.

The support portion 48 is configured so as to support (or hold) respective projection members arranged so as to be projected from the distal end face of the distal end portion 6b. Specifically, the support portion 48 is configured so as to support (or hold) a nozzle portion 49 for the front view observation window configured to eject a gas or liquid for cleaning the front view observation window 42, a front view illumination window 51 configured to emit the light for illuminating the front view direction, and a nozzle portion 52 for the side view observation window configured to eject a gas or liquid for cleaning the side view observation window side view observation window 43, as the above-described respectively projection members, respectively.

In the meantime, the support portion 48 is formed including a shield portion 48a which is an optical shield member for preventing acquisition of a side view field image including any one of the respective projection members due to appearance within the side view field of the respective projection members which are objects different from the original observation object. That is, by providing the shield portion 48a on the support portion 48, the side view field image not including any of the nozzle portion 49 for the front view observation window, the front view illumination window 51 and the nozzle portion 52 for the side view observation window can be obtained.

The nozzle portion 52 for the side view observation window is provided on two parts of the support portion 48 as illustrated in FIG. 11 and FIG. 12, and is arranged such that the distal end is projected to the side face of the support portion 48.

As illustrated in FIG. 14, the image pickup device 63 is electrically connected to the image processing portion 32a1, and outputs the front view field image and the side view field image picked up in the image pickup device 63 to the image processing portion 32a1. In addition, the inclination angle detection portion 17 is electrically connected to the image processing portion 32a1, and as illustrated in FIG. 15, detects the inclination angle of the rotating direction around the axis to the gravity direction of the insertion portion 4, with the axis being the longitudinal direction of the insertion portion 4, when a user twists the insertion portion 4 for example, and outputs the detected inclination angle information (inclination information) to the image processing portion 32a1.

The video processor 32 outputs drive signals for driving the image pickup device 63 provided on the distal end portion 6b of the endoscope 2. Then, the image processing portion 32a1 of the video processor 32 generates video signals by executing predetermined signal processing to image pickup signals outputted from the image pickup device 63 as the image signal generation portion, more specifically, generates an observation image including a front view field image forming a circular shape and a side view field image forming the circular ring shape on an outer periphery of the image in the front view direction, namely, the image for which the side view field image is arranged so as to surround the front view field image in the state that the side view field image is adjacent to the front view field image.

The boundary regions of the front view field image and the side view field image may overlap or may not overlap, and in the state that the boundary regions overlap, the front view observation window 42 and the side view observation window 43 may acquire partially overlapping object images, and the processing of partially removing the overlapping region may be performed in the image processing portion 32a1.

Then, the image processing portion 32a1 generates the image signals to which image processing of rotating the front view field image and the side view field image following the inclination angle based on the inclination angle information (inclination information) of the insertion portion 4 detected by the inclination angle detection portion 17, that is, the image processing of changing the arrangement angle of the direction where the field images are arranged and representing the state that the first object image and the second object image are inclined by relatively rotating the second object image to a part where the first object image is arranged, is executed, and outputs the image signals to the image output portion 32b. Note that, in the present embodiment, the image processing portion 32a1 rotates both of the front view field image and the side view field image according to the inclination angle of the insertion portion 4; however, only the front view field image or the side view field image may be rotated.

The image output portion 32b generates signals to be displayed on the monitor 35 from the image signals generated by the image processing portion 32a1, and outputs the signals to the monitor 35. Thus, the observation image including the front view field image forming the circular shape and the side view field image forming the circular ring shape on the outer periphery of the image in the front view direction is rotated according to the inclination angle of the insertion portion 4 and displayed on the monitor 35.

For example, when just one or more side view field images are arranged next to the front view field image, a perspective effect and a stereoscopic effect cannot be obtained, and it is difficult to recognize the image as an image of observing inside of a lumen without a feeling of incompatibility.

In contrast, by a display method of the front view field image and the side view field image in the modification, since setting is performed to be an optical structure that a screen radially spreads from the center to the periphery (such an optical characteristic is automatically attained by a lens in a circular ring shape) the perspective effect and the stereoscopic effect can be relatively easily obtained.

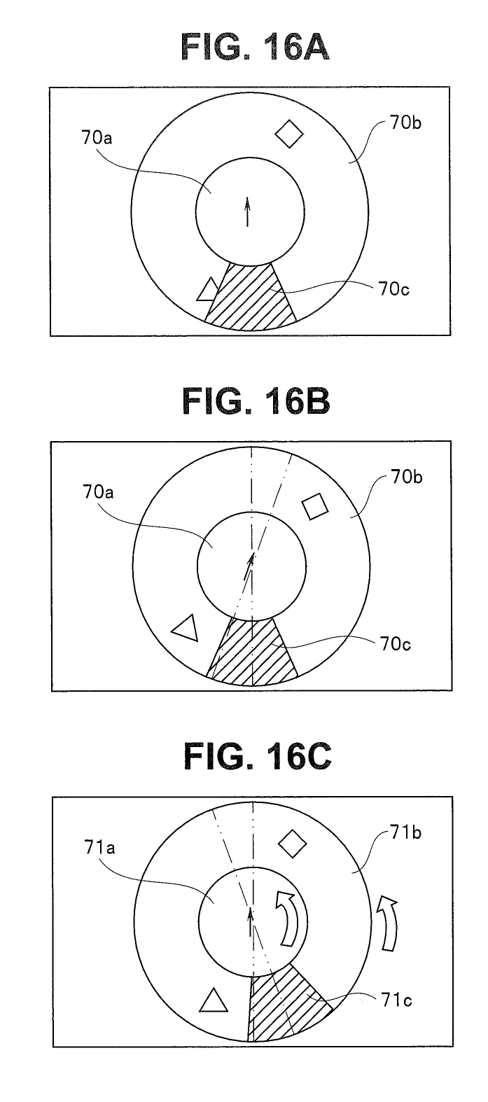

Next, the image processing by the image processing portion 32a1 will be described using FIG. 16A to FIG. 16C. FIG. 16A to FIG. 16C are diagrams illustrating one example of the observation images displayed on the monitor by the image processing by the image processing portion 32a1.

The image processing portion 32a1 acquires a front view field image 70a based on the first object image forming the circular shape acquired at the front view observation window 42, and a side view field image 70b based on the second object image forming the circular ring shape on the outer periphery of the first object image acquired at the side view observation window 43. Note that the side view field image 70b includes a shield region 70c optically shielded by the shield portion 48a of the support portion 48. Then, as illustrated in FIG. 16A, the image processing portion 32a1 generates the observation image for which the side view field image 70b forming the circular ring shape is formed on the outer periphery of the front view field image 70a forming the circular shape.

Here, when the insertion portion 4 of the endoscope 2 is twisted as illustrated in FIG. 15, as illustrated in FIG. 16B, the view point of the image pickup device 63 provided on the distal end portion 6a of the insertion portion 4 is also rotated around the axis in the longitudinal direction of the insertion portion 4 and the observation image is rotated so that it becomes difficult for a user to perform observation.

Therefore, the image processing portion 32a1 generates a front view field image 71a and a side view field image 71b (including a shield region 71c) for which the front view field image 70a and the side view field image 70b are rotated following the inclination angle of the insertion portion 4 as illustrated in FIG. 16C, based on the inclination angle information (inclination information) from the inclination angle detection portion 17.

In such a manner, the endoscope system 1 acquires the front view field image 70a by the front view observation window 42, acquires the side view field image 70b by the side view observation window 43, and arranges the side view field image 70b in the circular ring shape on the outer periphery of the front view field image 70a in the circular shape. Then, the endoscope system 1 generates the front view field image 71a and the side view field image 71b for which the front view field image 70a and the side view field image 70b are rotated based on the inclination angle information (inclination information) from the inclination angle detection portion 17.

As a result, since the front view field image 71a and the side view field image 71b rotated according to the rotating operation of the insertion portion 4 are displayed on the monitor 35, a user can recognize the inclination and the rotating angle or the like of the insertion portion 4.

Thus, according to the endoscope system of the present embodiment, similarly to the first embodiment, a range of performing the observation when the rotating operation of the endoscope or the like is performed can be easily recognized.

### Third Embodiment

Next, the third embodiment will be described.

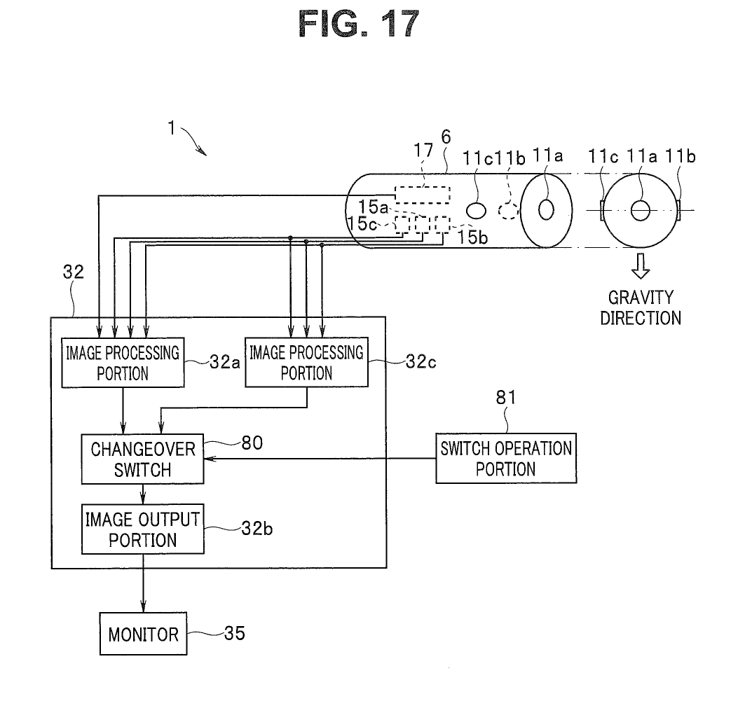

FIG. 17 is a diagram illustrating a configuration of a principal part in the third embodiment. Note that, in FIG. 17, for components similar to FIG. 4, same signs are attached and the description is omitted. As illustrated in FIG. 17, the video processor 32 of the present embodiment is configured by adding an image processing portion 32c and a changeover switch 80 to the video processor 32 (see FIG. 4) of the first embodiment.

To the image processing portion 32a, the front view field image 18a acquired by the front view observation window 11a, the side view field images 18b and 18c acquired by the side view observation windows 11b and 11c, and the inclination angle information (inclination information) of the insertion portion 4 detected by the inclination angle detection portion 17 are inputted. The image processing portion 32a which is operated also as the image signal generation portion arranges the front view field image 18a at the center, arranges the side view field images 18b and 18c on the left and right of the front view field image 18a, and then generates the front view field image 19a and the side view field images 19b and 19c for which the front view field image 18a and the side view field images 18b and 18c are rotated following the inclination angle of the insertion portion 4 based on the inclination angle information (inclination information) from the inclination angle detection portion 17. Then, the image processing portion 32a outputs the generated front view field image 19a and side view field images 19b and 19c to the changeover switch 80.

In the meantime, to the image processing portion 32c, the front view field image 18a acquired by the front view observation window 11a and the side view field images 18b and 18c acquired by the side view observation windows 11b and 11c are inputted. The image processing portion 32c which is operated also as the image signal generation portion arranges the front view field image 18a at the center, arranges the side view field images 18b and 18c on the left and right of the front view field image 18a, and outputs the images to the changeover switch 80.

To the changeover switch 80, switching signals from a switch operation portion 81 are inputted. The switch operation portion 81 is, for example, a switch provided on the operation portion 3 of the endoscope 2, a switch provided on the video processor 32, or a foot switch.

The changeover switch 80 outputs either output from the image processing portion 32a or output from the image processing portion 32c to the image output portion 32b, according to the switching signals from the switch operation portion 81. That is, the observation image (see FIG. 6B) to which rotating processing is not performed or the observation image (see FIG. 6C) to which the rotating processing is performed is outputted to the image output portion 32b from the changeover switch 80 according to the switching signals, and displayed on the monitor 35.

In such a manner, the endoscope system 1 of the present embodiment selects either one of the observation image to which the rotating processing is performed and the observation image to which the rotating processing is not performed by the changeover switch 80 and displays the image on the monitor 35. As a result, a user can select an optimum display mode according to an operating situation or the like of the endoscope 2 by switching ON/OFF of the rotating processing.

### Fourth Embodiment

Next, the fourth embodiment will be described.

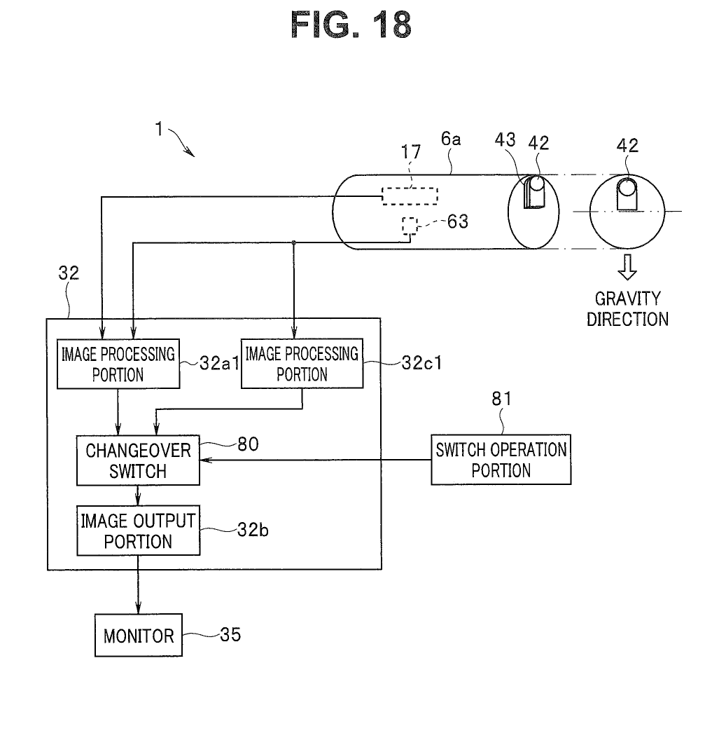

FIG. 18 is a diagram illustrating a configuration of a principal part in the fourth embodiment. Note that, in FIG. 18, for components similar to FIG. 14 and FIG. 17, same signs are attached and the description is omitted. As illustrated in FIG. 18, the video processor 32 of the present embodiment is configured by adding an image processing portion 32c1 and the changeover switch 80 to the video processor 32 (see FIG. 14) of the second embodiment.

To the image processing portion 32a1, the front view field image 70a acquired by the front view observation window 42, the side view field image 70b acquired by the side view observation window 43, and the inclination angle information (inclination information) of the insertion portion 4 detected by the inclination angle detection portion 17 are inputted. The image processing portion 32a1 which is operated also as the image signal generation portion arranges the side view field image 70b in the circular ring shape on the outer periphery of the front view field image 70a in the circular shape, and then generates the front view field image 71a and the side view field image 71b for which the front view field image 70a and the side view field image 70b are rotated based on the inclination angle information (inclination information) from the inclination angle detection portion 17. Then, the image processing portion 32a1 outputs the generated front view field image 71a and side view field image 71b to the changeover switch 80.

In the meantime, to the image processing portion 32c1, the front view field image 70a acquired by the front view observation window 42 and the side view field image 70b acquired by the side view observation window 43 are inputted. The image processing portion 32c1 which is operated also as the image signal generation portion arranges the side view field image 70b in the circular ring shape on the outer periphery of the front view field image 70a in the circular shape, and outputs the images to the changeover switch 80.

The changeover switch 80 outputs either output from the image processing portion 32a1 or output from the image processing portion 32c1 to the image output portion 32b, according to the switching signals from the switch operation portion 81. That is, the observation image (see FIG. 16B) to which the rotating processing is not performed or the observation image (see FIG. 16C) to which the rotating processing is performed is outputted to the image output portion 32b from the changeover switch 80 according to the switching signals, and displayed on the monitor 35.

According to such an endoscope system 1 of the present embodiment, similarly to the third embodiment, a user can select an optimum display mode according to an operating situation or the like of the endoscope 2 by switching ON/OFF of the rotating processing.

### Fifth Embodiment

Next, the fifth embodiment will be described.

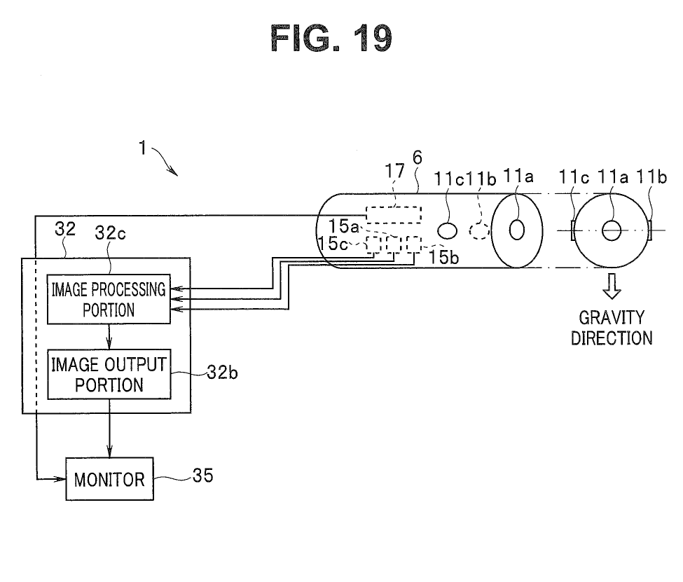

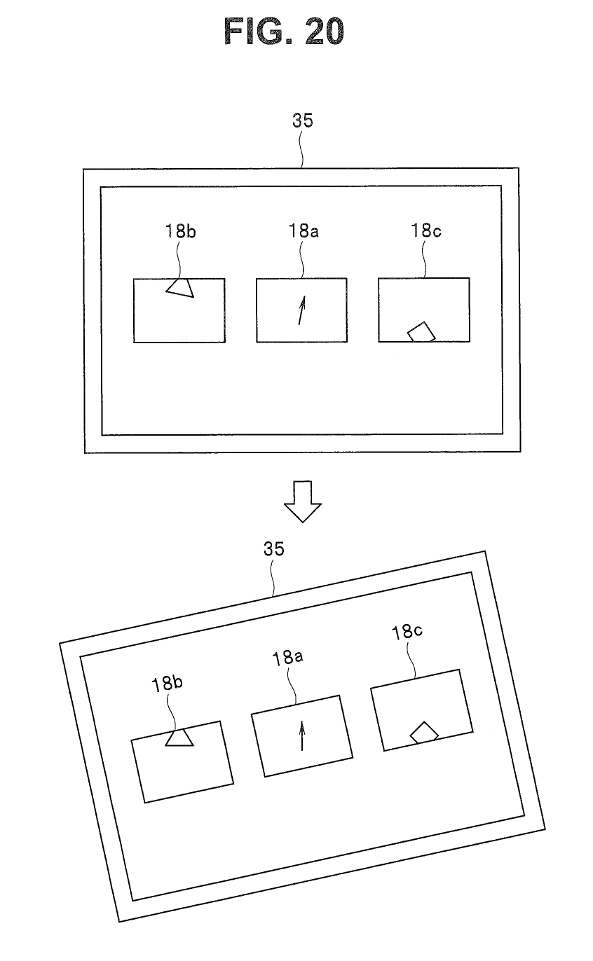

FIG. 19 is a diagram illustrating a configuration of a principal part in the fifth embodiment, and FIG. 20 is a diagram for illustrating a rotating operation of the monitor 35. Note that, in FIG. 19, for components similar to FIG. 4 and FIG. 17, same signs are attached and the description is omitted.

As illustrated in FIG. 19, to the image processing portion 32c, the front view field image 18a acquired by the front view observation window 11a, and the side view field images 18b and 18c acquired by the side view observation windows 11b and 11c are inputted. The image processing portion 32c which is operated also as the image signal generation portion arranges the front view field image 18a at the center, arranges the side view field images 18b and 18c on the left and right of the front view field image 18a, and outputs the images to the image output portion 32b.

In the meantime, the inclination angle information (inclination information) detected by the inclination angle detection portion 17 is inputted through the video processor 32 to the monitor 35. The monitor 35 has a rotation control portion and a rotating mechanism not shown in the figure, and the monitor 35 itself is rotated following the inclination angle of the insertion portion 4 as illustrated in FIG. 20, according to the inclination angle information (inclination information) from the inclination angle detection portion 17.

In such a manner, the endoscope system 1 of the present embodiment, the front view field image 18a and the side view field images 18b and 18c displayed on the monitor 35 are not rotated and the monitor 35 itself is rotated. As a result, according to the endoscope system 1 of the present embodiment, the range of performing the observation when the rotating operation of the endoscope or the like is performed can be easily recognized, similarly to the first embodiment.

Among the respective embodiments described above, in the first embodiment and the respective modifications, a mechanism of achieving a function of illuminating and observing the side is incorporated in the distal end portion 6 of the insertion portion 4 together with a mechanism of achieving a function of illuminating and observing the front; however, the mechanism of achieving the function of illuminating and observing the side may be a separate body attachable and detachable to/from the insertion portion 4.

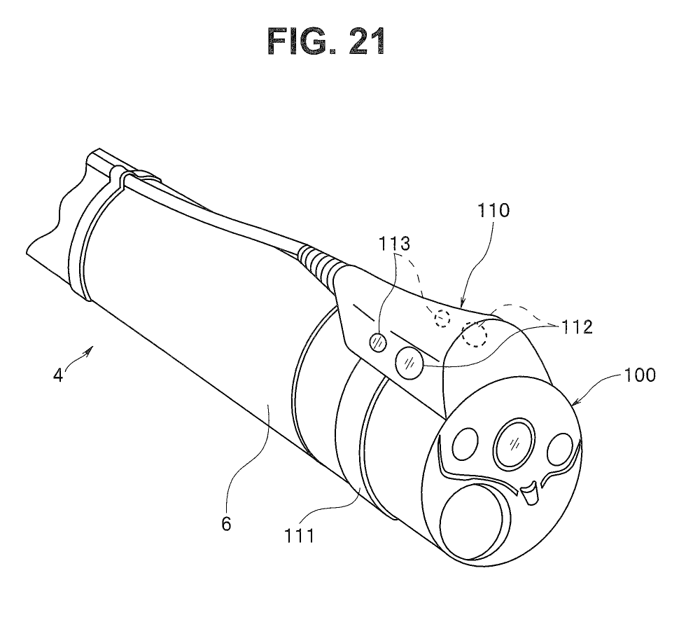

FIG. 21 is a perspective view of the distal end portion 6 of the insertion portion 4 to which a unit for the side observation is attached. The distal end portion 6 of the insertion portion 4 includes a unit 100 for a front view field. A unit 110 for a side view field has a configuration freely attachable and detachable to/from the unit 100 for the front view field by a clip portion 111.

The unit 110 for the side view field includes two observation windows 112 for acquiring images in left and right directions, and two illumination windows 113 for illuminating the left and right directions.

The video processor 32 or the like can perform acquisition of the observation image as indicated in the above-described first embodiment and display of arranging and displaying a plurality of screens on the monitor 35, by lighting and putting out the respective illumination windows 113 of the unit 110 for the side view field in accordance with a frame rate of the front view field.

The present invention is not limited to the above-described embodiments, and can be variously modified and altered or the like in the range of not changing the gist of the present invention.

What is claimed is:

1. An endoscope system comprising:
an insertion portion configured to be inserted inside a subject;
a first object image acquisition portion provided in the insertion portion, and configured to acquire a first object image from a first region of an object inside the subject including an insertion portion front part roughly parallel to a longitudinal direction of the insertion portion;
a first image pickup device configured to pick up the first object image and generate a first image pickup signal;
a second object image acquisition portion provided in the insertion portion, and configured to acquire a second object image from a second region of the object different from the first region, the second region including an insertion portion side part in a direction crossing the longitudinal direction of the insertion portion;
a second image pickup device configured to pick up the second object image and generate a second image pickup signal;
an inclination detection portion configured to detect a rotation around an axis, with the axis being the longitudinal direction of the insertion portion, and generate inclination information; and
an image signal generation portion configured to generate an image signal representing a state that an image represented by the second image pickup signal is made to revolve following the rotation and arranged with respect to an image represented by the first image pickup signal, based on the inclination information, the first image pickup signal and the second image pickup signal, and output the image signal to a display portion configured to display the image.

2. The endoscope system according to claim 1,
wherein the image signal generation portion generates the image signal such that the image generated by the first image pickup signal and the image generated by the second image pickup signal are arranged adjacently on a display screen.

3. The endoscope system according to claim 1,
wherein the image signal generation portion generates the image signal representing a state that the image represented by the second image pickup signal is made to autorotate following the rotation and arranged.

4. The endoscope system according to claim 3,
wherein the image signal generation portion generates the image signal representing a state that the image represented by the first image pickup signal is made to autorotate following the rotation and arranged.

5. The endoscope system according to claim 1,
wherein the image signal generation portion generates the image signal representing a state that only the image represented by the second image pickup signal is made to revolve following the rotation and arranged with respect to the image represented by the first image pickup signal, arrangement of which is fixed.

6. The endoscope system according to claim 1,
wherein the image signal generation portion switches
a mode of generating the image signal representing a state that the image represented by the second image pickup signal is made to only revolve following the rotation and arranged with respect to the image represented by the first image pickup signal, and
a mode of generating the image signal representing a state that, while the image represented by the second image pickup signal is made to autorotate following the rotation, the image represented by the second image pickup signal is made to revolve following the rotation and arranged with respect to the image represented by the first image pickup signal.

7. The endoscope system according to claim 1, further comprising
the display portion.

8. The endoscope system according to claim 7,
wherein the image signal generation portion outputs respective image signals for which an overlapping region of the image represented by the first image pickup signal and the image represented by the second image pickup signal is removed to the display portion.

9. The endoscope system according to claim 1,
wherein the second object image acquisition portion in plurality are arranged at roughly equal angles in a circumferential direction of the insertion portion, and
the image signal generation portion arranges the image represented by the first image pickup signal at a center, and arranges the image represented by the second image pickup signal in plurality at roughly equal angles in a circumferential direction of the image represented by the first image pickup signal.
